(19) United States
(12) Patent Application Publication
(10) Pub. No.: US 2026/0219108 A1
(43) Pub. Date: Jul. 30, 2026

(54) SYSTEMS AND METHODS TO ANALYZE SPECTROSCOPY DATA FOR MULTI-PATHOGEN DETECTION AND IDENTIFICATION

(71) Applicant: HyperSpectral Corp., Alexandria, VA (US)

(72) Inventors: Simi GEORGE, Alexandria, VA (US); Matthew THEURER, Alexandria, VA (US); Sarah Rachel HERNANDEZ, Austin, TX (US); Charles FREEMAN, Alexandria, VA (US)

(73) Assignee: HyperSpectral Corp., Alexandria, VA (US)

(21) Appl. No.: 19/457,616

(22) Filed: Jan. 23, 2026

Related U.S. Application Data
(60) Provisional application No. 63/749,484, filed on Jan. 24, 2025.

(51) Int. Cl. G01J 3/28; G01J 3/44; G01N 21/65; G01N 33/569

(52) U.S. Cl. CPC G01J 3/2823; G01J 3/44; G01N 21/65; G01N 33/569; G01J 2003/2826; G01N 2201/1296

(57) ABSTRACT

A non-transitory, processor-readable medium stores instructions that, when executed by a processor, cause the processor to provide Raman spectrum data as input to a 1-dimensional encoder to identify a feature of the Raman spectrum data. The instructions further cause the processor to provide hyperspectral image data as input to a 2-dimensional+1-dimesional encoder to identify a feature of the hyperspectral image data. Cross-modal attention is performed to (1) analyze, based on the feature of the Raman spectrum data, a region represented by the hyperspectral image data to identify a refined feature of the hyperspectral image data and (2) analyze, based on the feature of the hyperspectral image data, a peak represented by the Raman spectrum data to identify a refined feature of the Raman spectrum data. A type associated with a sample is classified based on the refined features of the hyperspectral image data and Raman spectrum data.

### CROSS REFERENCE TO RELATED APPLICATIONS

This application claims priority to and the benefit of U.S. Provisional Application No. 63/749,484, filed Jan. 24, 2025, and titled “SYSTEMS AND METHODS OF MODELING MULTI-PATHOGEN DETECTION IDENTIFICATION,” which is incorporated herein by reference.

### FIELD

One or more embodiments described herein relate to pathogen detection and identification and, more specifically, to systems and methods configured to use multi-modal machine learning models for detecting, identifying, characterizing, and quantifying multiple pathogens using spectroscopic and image data across distributed computing environments.

### BACKGROUND

Rapid, accurate, and scalable pathogen detection can be important in domains such as, for example, healthcare diagnostics, agriculture, food safety, biodefense, environmental monitoring, and/or etc. Some known laboratory techniques, such as polymerase chain reaction (PCR), culturing, and immunoassays, offer sensitivity but are constrained by cost, infrastructure requirements, processing time, and limited suitability for continuous and/or in-field monitoring. Some known spectroscopic and/or imaging-based sensing techniques generate rich, high-dimensional data but are highly susceptible to confounding factors including, for example, background substrates, mixed biological signatures, environmental noise, sensor drift, illumination variation, and/or cross-instrument variability. These effects are amplified when multiple pathogens coexist and/or when a pathogen is present at low abundance. Some known single-model analytical pipelines lack sufficient robustness and generalization. A need exists, therefore, for systems and methods that are broadly applicable and that leverage model diversity, redundancy, adaptive integration, and/or distributed computation to suppress confounders and enable reliable multi-pathogen detection under real-world conditions.

### SUMMARY

According to an embodiment, a non-transitory, processor-readable medium stores instructions that, when executed by a processor, cause the processor to provide Raman spectrum data associated with a sample as input to a 1-dimensional encoder to identify a feature of the Raman spectrum data. The instructions further cause the processor to provide hyperspectral image data associated with the sample as input to a 2-dimensional+1-dimesional encoder to identify a feature of the hyperspectral image data. Cross-modal attention is performed to (1) analyze, based on the feature of the Raman spectrum data, a region represented by the hyperspectral image data to identify a refined feature of the hyperspectral image data and (2) analyze, based on the feature of the hyperspectral image data, a peak represented by the Raman spectrum data to identify a refined feature of the Raman spectrum data. A type associated with the sample is classified based on (1) the refined feature of the hyperspectral image data and (2) the refined feature of the Raman spectrum data.

According to an embodiment, a method includes providing, via a processor, first spectrum data associated with (1) a pathogen and (2) a first spectrum type as input to a 1-dimensional encoder to identify a feature of the first spectrum data. The method further includes providing, via the processor, second spectrum data associated with (1) the pathogen (2) a second spectrum type different from the first spectrum type as input to a 2-dimensional+1-dimesional encoder to identify a feature of the second spectrum data. Cross-modal attention is performed, via the processor, to (1) analyze, based on the feature of the first spectrum data, a region represented by the second spectrum data to identify a refined feature of the second spectrum data and (2) analyze, based on the feature of the second spectrum data, a peak represented by the first spectrum data to identify a refined feature of the first spectrum data. The method also includes classifying, via the processor, a cell type associated with the pathogen based on (1) the refined feature of the first spectrum data and (2) the refined feature of the second spectrum data.

### BRIEF DESCRIPTION OF THE DRAWINGS

FIG. 1 shows a system block diagram of a spectroscopy analysis system, according to an embodiment.

FIG. 2 shows a system block diagram of a compute device included in a spectroscopy analysis system, according to an embodiment.

FIG. 3 shows a system block diagram of spectroscopy analysis components included in a spectroscopy analysis system, according to an embodiment.

FIG. 4 shows a flow diagram illustrating a method for classifying a type associated with a sample based on refined features of Raman spectrum data and hyperspectral image data, according to an embodiment.

FIG. 5 shows a flow diagram illustrating a method for classifying a cell type associated with a pathogen based on refined features of first and second spectrum data, according to an embodiment.

### DETAILED DESCRIPTION

At least some systems and methods described herein are configured to at least one of detect, identify, characterize, and/or quantify multiple pathogens in the presence of confounding factors. In some implementations, pathogens can be counted by taking a spectral measurement of each cell, identifying a cell type for that cell based on the spectral reading, and incrementing a counter associated with that cell type. A cell type can include, for example, a bacteria type, a bacteria strain, an indication of resistance to a drug (e.g., an antibiotic), and/or the like. Alternatively or in addition, at least some systems and methods described herein are configured to analyze other samples, such as viruses, fungi, and/or other pathogens. A confounding factor can include, for example, objects (e.g., white blood cells, red blood cells, fibrin, gout crystals, microscopic pieces of dirt, intra-cellular debris, and/or etc.) of similar morphology (e.g., size, shape, and/or etc.) that can be mistaken for bacterial cell of interest. In some embodiments, sensor data from one or more modalities (e.g., associated with one or more spectrum types, such as a Raman spectrum and/or etc.) is processed through adaptive preprocessing pipelines, analyzed using multiple heterogeneous machine learning models, and integrated using dynamic voting, weighting, ranking, selection, and/or suppression mechanisms, as described herein. At least some systems and methods described herein involve centralized, edge, cloud, hybrid, and/or federated learning environments.

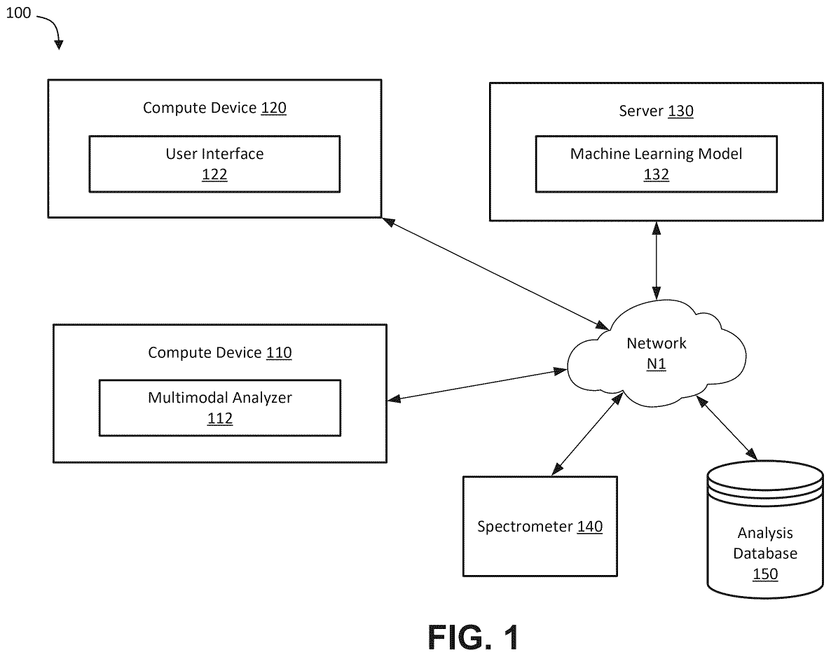

FIG. 1 shows a system block diagram of a spectroscopy analysis system 100, according to an embodiment. The spectroscopy analysis system 100 includes a compute device 110, a compute device 120, a server 130, a spectrometer 140, an analysis database 150, and a network N1. The spectroscopy analysis system 100 can include alternative configurations, and various steps and/or functions of the processes described below can be shared among the various devices of the spectroscopy analysis system 100 or can be assigned to specific devices (e.g., the compute device 110, the compute device 120, the server 130, and/or the like) different from the descriptions herein. For example, in some configurations, a user can provide inputs (as described herein) directly to the compute device 110 rather than via the compute device 120.

In some implementations, the compute device 110, the compute device 120, and/or the server 130 can include any suitable hardware-based computing devices and/or multimedia devices, such as, for example, a server, a desktop compute device, a smartphone, a tablet, a wearable device, a laptop and/or the like. In some implementations, the compute device 110, the compute device 120, and/or the server 130 can be implemented at an edge (e.g., with respect to the network N1) node or other remote (e.g., with respect to the network N1) computing facility and/or device. In some implementations, each of the compute device 110, the compute device 120, and/or the server 130 can be (or be included in) a data center or other control facility and/or device configured to run and/or execute a distributed computing system and can communicate with other compute devices.

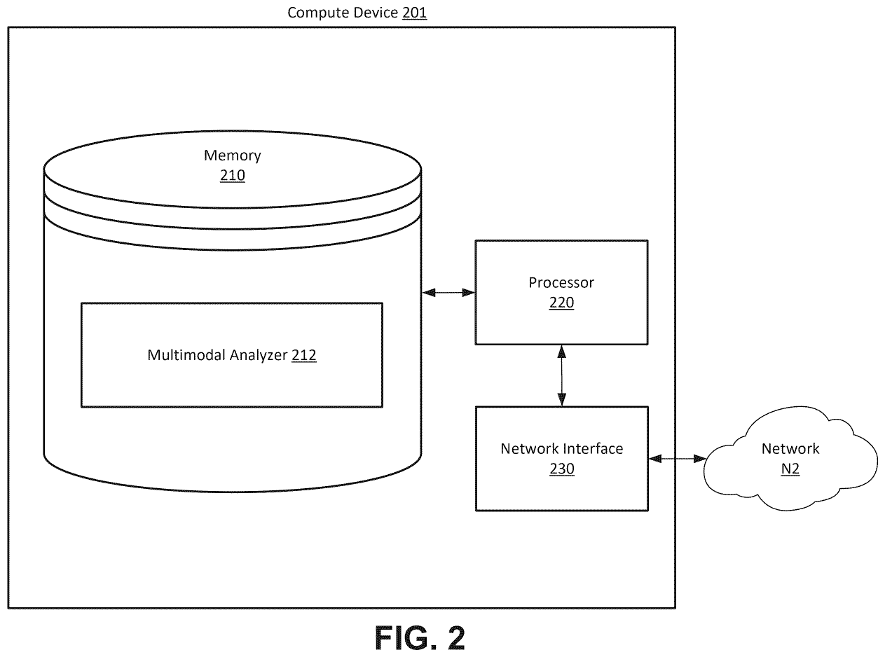

The compute device 110 can include a spectroscopy analyzer 112, which can include software (1) stored at a memory that is functionally and/or structurally similar to the memory 210 of FIG. 2 discussed below and (2) executed via a processor that is functionally and/or structurally similar to the processor 220 of FIG. 2 discussed below. The spectroscopy analyzer 112 can be configured to analyze spectra data produced by the spectrometer 140 (described herein) to, for example, use multiple heterogeneous machine learning models (including the machine learning model 132) to at least one of detect, identify, characterize, and/or quantify multiple pathogens, as described herein. The spectroscopy analyzer 112 can be functionally and/or structurally similar to the spectroscopy analyzer 212 of FIG. 2.

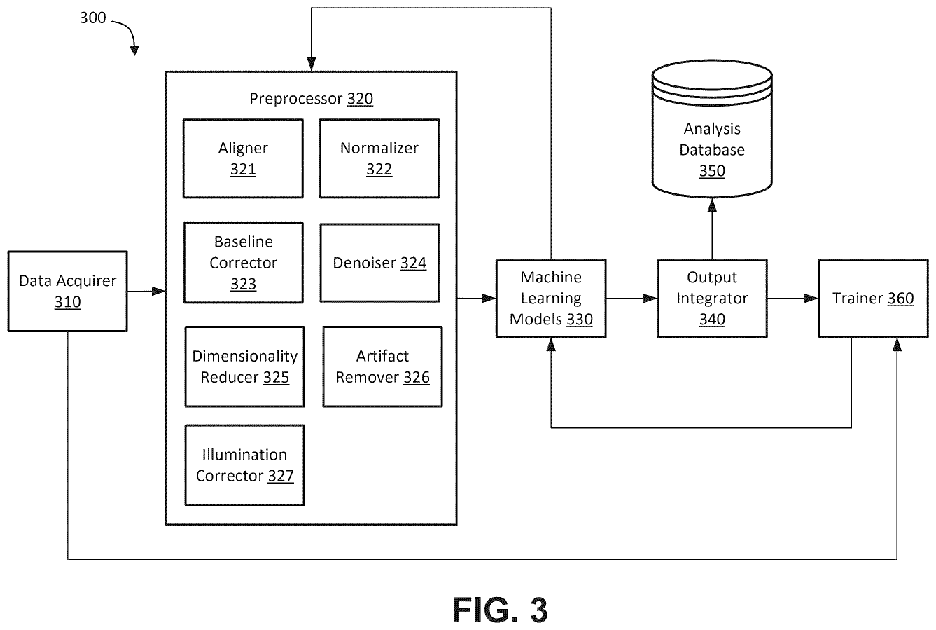

The compute device 120 can implement a user interface 122, which can include a programmatic interface (e.g., an application programming interface (API), a graphical user interface (GUI) (e.g., displayed on a monitor/display), and/or etc.) that is configured to receive input data (e.g., similar to the input data 302 of FIG. 3) from a user. The user interface 122 can further cause return and/or display of output data generated by the spectroscopy analyzer 112 (e.g., based on persistent data produced by the spectroscopy analyzer 112, as described herein). The user interface 122 can be implemented via software and/or hardware.

The server 130 can include a remote (e.g., as to the compute device 110 and/or the compute device 120) compute device(s) that can be configured to train, host, and/or execute a machine learning model 132. The machine learning model 132 can be functionally and/or structurally similar to at least one machine learning model from the machine learning models 330 of FIG. 3 (described herein). The machine learning model 132 can include, for example, a feedforward neural network, a convolutional neural network, a support vector machine (SVM), a random forest, gradient boosted decision trees, an autoencoder, a Siamese neural network, and/or etc., as described further herein. In some implementations, the compute device 110 can execute a service (e.g., a prompt service) to provide input data to the machine learning model 132. Alternatively or in addition, although not shown in FIG. 1, the compute device 110 can train, host, and/or execute the machine learning model 132.

The spectrometer 140 can be configured to perform spectroscopy (e.g., Raman spectroscopy, Fourier transform infrared spectroscopy (FTIR), and/or etc.), spectrometry, hyperspectral imaging, (HSI), and/or the like, to produce spectrum data, as described herein. The spectrometer 140 can be associated with the data acquirer 310 of FIG. 3, each described herein.

The analysis database 150 can store results (e.g., sample profile predictions) produced by the spectroscopy analyzer 112 and/or the machine learning model 132. The analysis database 150 can be functionally and/or structurally similar to the analysis database 350 of FIG. 3, described herein.

The compute device 110 can be networked and/or communicatively coupled to the compute device 120, the server 130, the spectrometer 140, and/or the analysis database 150, via the network N1, using wired connections and/or wireless connections. The network N1 can include various configurations and protocols, including, for example, short range communication protocols, Bluetooth®, Bluetooth® LE, the Internet, World Wide Web, intranets, virtual private networks, wide area networks, local networks, private networks using communication protocols proprietary to one or more companies, Ethernet, WiFi® and/or Hypertext Transfer Protocol (HTTP), cellular data networks, satellite networks, free space optical networks and/or various combinations of the foregoing. Communication can be facilitated by any device capable of transmitting data to and from other compute devices, such as a modem(s) and/or a wireless interface(s).

In some implementations, although not shown in FIG. 1, the spectroscopy analysis system 100 can include multiple compute devices 110, compute devices 120, and/or servers 130. For example, in some implementations, the spectroscopy analysis system 100 can include multiple compute devices 110, where each compute device 110 can be associated with a different user from multiple users. In some implementations, multiple compute devices 110 can be associated with a single user, where each compute device 110 can be associated with, for example, a different input modality (e.g., text input, audio input, analog and/or digital signal input, image input, video input, etc.). Some implementations can include various combinations of the above.

FIG. 2 shows a system block diagram of a compute device 201 included in a spectroscopy analysis system, according to an embodiment. The compute device 201 can be structurally and/or functionally similar to, for example, the compute device 110 of the spectroscopy analysis system 100 shown in FIG. 1. The compute device 201 can be a hardware-based computing device, a multimedia device, or a cloud-based device such as, for example, a computer device, a server, a desktop compute device, a laptop, a smartphone, a tablet, a wearable device, a remote computing infrastructure, and/or the like. The compute device 201 includes a memory 210, a processor 220, and a network interface 230 operably coupled to a network N2.

The processor 220 can be, for example, a hardware-based integrated circuit (IC), or any other suitable processing device configured to run and/or execute a set of instructions or code (e.g., stored in memory 210). For example, the processor 220 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a graphics processing unit (GPU), a programmable logic controller (PLC), a remote cluster of one or more processors associated with a cloud-based computing infrastructure and/or the like. The processor 220 is operatively coupled to the memory 210. In some implementations, for example, the processor 220 can be coupled to the memory 210 through a system bus (for example, address bus, data bus and/or control bus). In some implementations, the processor 220 can include multiple parallelly arranged processors.

The memory 210 can be, for example, a random-access memory (RAM), a memory buffer, a hard drive, a read-only memory (ROM), an erasable programmable read-only memory (EPROM), and/or the like. The memory 210 can store, for example, one or more software modules and/or code that can include instructions to cause the processor 220 to perform one or more processes, functions, and/or the like. In some implementations, the memory 210 can be a portable memory (e.g., a flash drive, a portable hard disk, and/or the like) that can be operatively coupled to the processor 220. In some instances, the memory can be remotely operatively coupled with the compute device 201, for example, via the network interface 230. For example, a remote database server can be operatively coupled to the compute device 201.

The memory 210 can store various instructions associated with processes, algorithms and/or data, as described herein. Memory 210 can further include any non-transitory computer-readable storage medium for storing data and/or software that is executable by processor 220, and/or any other medium, which may be used to store information that may be accessed by processor 220 to control the operation of the compute device 201. For example, the memory 210 can store data associated with a spectroscopy analyzer 212. The spectroscopy analyzer 212 can be configured to analyze spectra data to, for example, use multiple heterogeneous machine learning models to at least one of detect, identify, characterize, and/or quantify multiple pathogens, as described herein. The spectroscopy analyzer 212 can be functionally and/or structurally similar to the spectroscopy analyzer 112 of FIG. 1.

The network interface 230 can be configured to connect to the network N2, which can be functionally and/or structurally similar to the network N1 of FIG. 1. For example, network N2 can use any of the communication protocols described above with respect to network N1 of FIG. 1. In some implementations, the network interface 230 can include a network interface controller (NIC) that implements a physical and/or data link layer (e.g., Ethernet, Wi-Fi®, etc.).

In some instances, the compute device 201 can further include a display, an input device, and/or an output interface (not shown in FIG. 2). The display can be any display device (e.g., a monitor, screen, etc.) by which the compute device 201 can output and/or display data (e.g., via a user interface that is structurally and/or functionally similar to the user interface 122 of FIG. 1). The input device can include, for example, a mouse, keyboard, touch screen, voice interface, and/or any other hand-held controller or device or interface via which a user may interact with the compute device 201. The output interface can include, for example, a bus, port, and/or other interfaces by which the compute device 201 may connect to and/or output data to other devices and/or peripherals.

FIG. 3 shows a system block diagram of spectroscopy analysis components 300 included in a spectroscopy analysis system, according to an embodiment. At least a portion of the spectroscopy analysis components 300 can be associated with a compute device (e.g., a compute device that is structurally and/or functionally similar to the compute device 201 of FIG. 2 and/or the compute devices 110 and 120 of FIG. 1). For example, the spectroscopy analysis components 300 can include, be included in, implement, and/or be associated with (1) the spectroscopy analyzer 112 of FIG. 1 and/or (2) the spectroscopy analyzer 212 of FIG. 2. In some instances, the spectroscopy analysis components 300 can include software stored in memory 210 and configured to execute via the processor 220 of FIG. 2. In some instances, at least a portion of the spectroscopy analysis components 300 can be implemented in hardware (e.g., an ASIC) or a combination of hardware (e.g., a general-purpose processor) and software.

The spectroscopy analysis components 300 include a data acquirer 310, a preprocessor 320, machine learning models 330, an output integrator 340, an analysis database 350, and a trainer 360. The preprocessor 320 includes an aligner 321, a normalizer 322, a baseline corrector 323, a denoiser 324, a dimensionality reducer 325, an artifact remover 326, and an illumination corrector 327.

The data acquirer 310 can include hardware and/or software (e.g., an analog-to-digital converter (DAC)) configured to interface with once more sensors (e.g., a spectrometer(s) that is functionally and/or structurally similar to the spectrometer 140 of FIG. 1) to acquire spectroscopic and/or image data from samples. As described herein, this spectroscopic and/or image data can be preprocessed, and machine learning model outputs can be integrated to generate pathogen detection results. Processing of the spectroscopic and/or image data can occur locally, at the edge, in the cloud, and/or across distributed computing environments.

In some implementations, the data acquirer 310 can use the one or more sensors to analyze a sample that has been prepared by, for example, culturing target pathogens under standardized conditions (e.g., on agar or in broth) to minimize biological variability. Cells can then be washed and placed onto gold-coated slides to reduce extraneous fluorescence and ensure consistent surface interactions for Raman measurements. A 785 nm laser with a Raman spectrometer (e.g., a Wasatch Photonics (WP-785X-F13-R-ILC-785 nm 10C regulated Raman spectrometer) can be used with, for example, ~8 cm⁻¹ spectral resolution. This spectrometer can be functionally and/or structurally similar to the spectrometer 140 of FIG. 1. Each pathogen isolate can be sampled, for example, 10 or more times to capture within-strain heterogeneity.

The preprocessor 320 can be configured to perform preprocessing operations, including spectral alignment (via the aligner 321), normalization (e.g., area under the curve (AUC) normalization and/or the like, via the normalizer 322), baseline correction (e.g., an asymmetric least squares (ALS) baseline correction and/or the like, via the baseline corrector 323), denoising (via the denoiser 324), dimensionality reduction (via the dimensionality reducer 325), artifact removal (via the artifact remover 326), and illumination correction (via the illumination corrector 327). In some implementations, selection, sequencing, and/or parameterization of preprocessing steps can be dynamically controlled by the preprocessor 320 based on, for example, metadata, inferred noise characteristics, or model feedback.

Describing the aligner 321 in more detail, this component can be configured to select a reference baseline (b_{ref}(λ)) (e.g., a median spectrum and/or an estimated baseline). For each spectrum xᵢ(λ), the aligner 321 can estimate a baseline bᵢ(λ) (e.g., via a polynomial and/or spline technique) and compute an offset and/or affine transform xᵢ′(λ)=xᵢ(λ)−bᵢ(λ)+b_{ref}(λ). In some implementations, the aligner 321 can be configured to enforce smoothness and/or monotonicity constraints.

Alternatively or in addition, the aligner 321 can be configured to performed peak-anchored alignment of spectrum data by identifying invariant anchor regions (e.g., non-absorbing windows) and estimating a baseline offset using anchor points associated with the invariant anchor regions (and not other points). Based on this baseline offset, the aligner 321 can then interpolate correction across a full spectrum.

In some implementations, the aligner 321 can be configured to perform optimal (or improved) transport baseline matching by aligning low-frequency components using Wasserstein distance minimization (and/or the like). Alternatively or in addition, the aligner 321 can include (or have access to) a neural network(s) that includes learned alignment layers configured to learn baseline alignment jointly with classification (described herein), such that preprocessing parameters are treated as hyperparameters during training of the machine learning models 330.

Turning to the normalizer 322 in further detail, this component can remove scale differences in spectrum data due to, for example, concentration, path length, and/or illumination. More specifically, the normalizer 322 can perform vector normalization (e.g., by computing an L2 norm of a spectrum and dividing intensities of the spectrum by the L2 norm), area normalization, and/or computation of standard normal variate (SNV). Alternatively or in addition, the normalizer 322 can perform adaptive normalization (e.g., by normalizing only within chemically meaningful bands), learned normalization (e.g., using scale factors predicted via a neural network conditioned on spectrum statistics), and/or physics-aware normalization to enforce invariance to laser power while preserving relative peak ratios.

The baseline corrector 323 can, for example, remove slowly varying background (e.g., fluorescence in the Raman spectrum) by performing polynomial fitting and/or asymmetric least squares (ALS). In some implementations, the baseline corrector 323 can be configured to perform at least one of morphological baseline correction (e.g., using opening/closing operators), deep baseline estimation (e.g., using autoencoders trained to output baseline-only signals), and/or multi-scale baseline estimation (e.g., by separating fluorescence and scattering contributions at different scales).

Describing the denoiser 324 in further detail, this component can be configured to remove high-frequency noise while preserving spectral peaks, using, for example, Savitzky-Golay filtering by choosing a window size and polynomial order, fitting a local polynomial within the window, and replacing a center point with the polynomial value. Alternatively or in addition, the denoiser 324 can perform wavelet denoising by applying a discrete wavelet transform (DWT), determining threshold high-frequency coefficients, and performing an inverse transform. Alternatively or in addition, the denoiser 324 can perform principal component analysis (PCA) denoising by fitting PCA data to the spectrum data, retaining a predetermined number of top (e.g., best fitting) components, and reconstructing the spectrum. In some implementations, the denoiser 324 can be configured to (1) denoise spectral data using similar spectral neighborhoods of non-local spectra, (2) perform self-supervised denoising (e.g., Noise2Noise and/or Noise2Void), and/or (3) use physics-constrained autoencoders configured to preserve peak widths and positions within spectrum data.

The dimensionality reducer 325 can be configured to, for example, reduce data redundancy (e.g., to reduce memory usage) and/or improve generalization. More specifically, the dimensionality reducer 325 can be configured to perform (1) PCA (e.g., by determining mean-center data, computing a covariance matrix (e.g., represented by covariance matrix data) based on the mean-center data, performing eigen-decomposition based on the covariance matrix, and projecting the result on top-matching eigenvectors), (2) supervised partial least squares (PLS) regression (e.g., by maximizing covariance between spectra and labels, and extracting latent variables), and/or (3) autoencoder-based analysis (e.g., by encoding a spectrum to a latent vector, decoding the latent vector back to the spectrum, and training an autoencoder based on reconstruction loss). In some implementations, the dimensionality reducer 325 can address spectral attention bottlenecks by learning band importance, can perform manifold learning with physics constraints, and/or perform band-selection based on sparsity penalties (e.g., that are associated with L1/group Lasso).

The artifact remover 326 can be configured to, for example, remove non-chemical distortions (e.g., resulting from cosmic rays, dead pixels, spikes, and/or etc.) For spike/cosmic ray removal, the artifact remover 326 can detect outliers via large first and/or second derivatives and replace affected points via interpolation and/or local median. The artifact remover 326 can perform mask-based removal by identifying predefined artifact bands, and masking and/or interpolating over the predefined artifact bands. In some implementations, the artifact remover 326 can include (or have access to) a learned artifact detector (e.g., a CNN trained to flag spikes). Alternatively or in addition, the artifact remover 326 can be configured to at least one of (1) perform temporal consistency checks (e.g., using repeated measurements), (2) use robust loss functions that ignore transient outliers during training, and/or (3) replace affected points via interpolation or local median.

Turning to the illumination corrector 327, this component can be configured to compensate for non-uniform and/or drifting illumination intensity by performing at least one of reference-based correction, flat-field correction, and/or multiplicative scatter correction (MSC). In some implementations, the illumination corrector 327 can produce joint illumination-material models using bilinear factorization, promote illumination invariance by penalizing models that are sensitive to illumination scaling, and/or apply learned correction fields that are integrated as differentiable layers.

As described above, observed noise characteristics in the received spectrum data can provide a feedback signal to adapt the preprocessor 320. For example, the preprocessor 320 can detect high-frequency random noise (e.g., shot noise, detector noise, and/or etc.) based on rapid band-to-band fluctuations, poor repeatability between replicates, and/or flattened and/or unstable peak maxima. The preprocessor 320 can receive further feedback from the machine learning models 330 (described herein), including, for example, high prediction variance across replicates. unstable logits under small input perturbations, and/or attention/gradient maps scattered across bands. In response, the preprocessor 320 can facilitate at least one of parameter changes (e.g., increasing Savitzky-Golay (SG) window size (e.g., 7→15 bands), increasing wavelet threshold level, and/or etc.), step substitutions (e.g., replacing SG filter with wavelet denoising and/or PCA denoising), and/or sequence changes (e.g., by performing denoising earlier, before baseline correction if baseline fitting is overfitting noise). In some implementations, the preprocessor 320 can respond to high-frequency random noise by using a Noise2Noise denoising model that trained on replicate spectra to preserve peak shapes.

To detect low-frequency baseline drift (e.g., resulting from fluorescence, temperature effects, and/or etc.), the preprocessor 320 can detect curved or sloped baselines, peak asymmetry and/or elevated background, and/or day-to-day baseline variability. In some implementations, the machine learning models 330 (described herein) can provide further feedback of low-frequency baseline drift if, for example, the machine learning models 330 heavily rely on low-frequency components, the machine learning models 330 have good training accuracy but poor cross-day generalization, and/or feature importance predicted by the machine learning models 330 are concentrated outside predetermined biochemical peaks.

In response to detecting/receiving indication of low-frequency baseline drift, the preprocessor 320 can facilitate at least one of parameter changes (e.g., increase ALS smoothing parameter λ, increase asymmetry parameter p to penalize peak leakage, and/or etc.), step substitutions (e.g., replace polynomial baseline correction with ALS and/or morphological filtering) and/or sequencing changes (e.g., add baseline alignment after baseline correction and/or move normalization after baseline correction). In some implementations, the preprocessor 320 can perform a multi-scale baseline correction by subtracting two baselines with different smoothness.

To detect impulsive noise/cosmic spikes, the preprocessor 320 can detect isolated, extremely sharp peaks, non-reproducible across replicates, and/or saturation of single detector bins. In some implementations, the machine learning models 330 (described herein) can provide further feedback of impulsive noise/cosmic spikes if, for example, the machine learning models 330 heavily rely on low-frequency components, the machine learning models 330 have high confidence but poor/wrong predictions, saliency maps spike at single bands, and/or the machine learning models 330 have adversarial vulnerability to single-band perturbations.

In response to detecting/receiving indication of impulsive noise/cosmic spikes, the preprocessor 320 can facilitate at least one of parameter changes (e.g., lower spike detection threshold (e.g., by changing a median absolute deviation (MAD) multiplier from 8 to 5)), step substitutions (e.g., replace derivative thresholding with trained spike detector), and/or sequencing changes (e.g., ensure spike removal is performed before any smoothing or PCA). In some implementations, the preprocessor 320 can be configured to inject synthetic spikes (e.g., synthetically injected spike data) during training of the machine learning models 330, to train the machine learning models 330 to ignore the synthetic spikes. More specifically, to inject synthetic spikes into training data, the preprocessor 320 (and/or the trainer 360, described herein) can create realistic spike patterns by modeling underlying rates, adding noise, and/or combining with known spike shapes, using, for example, statistical distributions (e.g., Gamma distributions for intervals) and/or convolution with templates, ensuring similarity to real data characteristics, such as signal-to-noise ratio (SNR), overlap, and/or the like.

To detect multiplicative illumination/intensity variation, the preprocessor 320 can detect, among the spectrum data, common spectral shapes having different amplitudes, replicates that differ mainly by scale factor, and/or strong correlation between norm and class prediction. In some implementations, the machine learning models 330 (described herein) can provide further feedback of multiplicative illumination/intensity variation if, for example, confidence of the machine learning models 330 tracks total intensity and/or if classification breaks under laser power change.

In response to detecting/receiving indication of multiplicative illumination/intensity variation, the preprocessor 320 can facilitate at least one of parameter changes (e.g., switch from area normalization to SNV and/or L2 normalization), step substitutions (e.g., replace global normalization with MSC), and/or sequencing changes (e.g., apply illumination correction before baseline correction). In some implementations, the preprocessor 320 can be configured to perform adversarial intensity scaling during training of the machine learning models 330 to enforce invariance.

In some implementations, the preprocessor 320 can detect overfitting of the machine learning models 330 to an instrument and/or session. This detection can be based on, for example, strong training accuracy yet weak test accuracy on a new instrument, t-SNE/PCA clusters being based on acquisition date, and/or domain classifiers easily predicting instrument ID. In response, the preprocessor 320 can add steps (e.g., baseline spectral alignment and/or explicit instrument-wise normalization), alter parameters (e.g., more aggressive baseline smoothing and/or stronger normalization), and/or facilitate sequencing changes (e.g., insert alignment step between baseline correction and denoising). In some implementations, the preprocessor 320 can be configured to facilitate domain-adversarial training of the machine learning models 330 combined with preprocessing constraints.

In some implementations, the preprocessor 320 can detect, of the machine learning models 330, class confusion between chemically similar bacteria. This detection can be based on a confusion matrix shows symmetric misclassification, feature importance that highlights noisy and/or irrelevant bands, and/or a latent space that lacks separation. In response, the preprocessor 320 can alter steps (e.g., reduce denoising strength to preserve subtle peaks and/or normalize within fingerprint region only), alter parameters (e.g., reduce SG window size and/or lower PCA component count (to avoid noise dimensions)), and/or facilitate sequencing changes (e.g., move dimensionality reduction after normalization and alignment). In some implementations, the preprocessor 320 can be configured to reduce class confusion by performing band selection based on sparsity penalties.

In some implementations, the preprocessor 320 can detect, of the machine learning models 330, sensitivity to small perturbations (poor robustness). This detection can be based on adversarial attacks succeeding with small perturbation & (e.g., below a predetermined threshold), high gradient norms with respect to input, and/or prediction flips under ±1% noise. In response, the preprocessor 320 can add steps (e.g., perform stronger denoising and/or adversarial data augmentation), alter parameters (e.g., increase smoothing or wavelet thresholds), and/or facilitate sequencing changes (e.g., move denoising before normalization to avoid noise amplification). In some implementations, the preprocessor 320 can be configured to improve robustness by training preprocessing parameters (e.g., parameters associated with differentiable ALS, SG, and/or etc.) end-to-end with the machine learning models 330.

To illustrate an example feedback response of the preprocessor 320, the preprocessor 320 can detect poor cross-day performance based on trained accuracy being 98% one day and 72% the next day. In response, the preprocessor 320 can infer baseline drift and illumination variation and, to correct the drift and variation, can increase ALS λ by 10×, add baseline alignment step and switch normalization from area to SNV.

As yet another example feedback response of the preprocessor 320, the preprocessor 320 can detect high confidence wrong predictions based on the machine learning models 330 exhibiting high confidence on misclassified spectra, which the preprocessor 320 can diagnose based on cosmic spikes dominating predicted features. In response, the preprocessor 320 can perform derivative-based spike removal, can lower a spike detection threshold, and/or can facilitate retraining of the machine learning models 330 with spike-augmented data.

As further illustration, the preprocessor 320 can detect disagreement between replicates based on the machine learning models 330 producing different predictions for the same sample, which the preprocessor 320 can diagnose as being the result of the presence of high-frequency noise and/or inconsistent smoothing. In response, the preprocessor 320 can replace SG filtering with wavelet denoising, apply denoising earlier in the pipeline, and/or increase PCA denoising strength.

The machine learning models 330 can include a plurality of heterogeneous models, including, for example, decision-tree-based models, deep neural networks (e.g., convolutional neural networks (CNNs)), metric learning models (e.g., configured to automatically define task-specific distance metrics from supervised data (e.g., weakly supervised data), where the metric can then be used to perform classification tasks), probabilistic models, ensemble learners, autoencoders, and/or self-supervised and/or contrastive learning models. In some instances, the machine learning models can be specialized/configured based on, for example, modality, pathogen class, geography, and/or operating conditions.

In some implementations, unlike some known CNNs that are configured to assume spatial locality, the machine learning models 330 can include a CNN that is configured to consider ordered chemical continuity. For example, for Raman/1-dimensional (1D) spectra data, the machine learning models 330 can use 1D convolutions with small kernels (e.g., 3-9 wavelength bands, each having a predefined width) for fine and/or single peak structure and larger kernels (e.g., 15-51 bands) for broad features (e.g., that span multiple peaks, such as a periodicity feature, a trough feature, and/or other multi-peak features). In some implementations, the CNN can be configured to perform multi-scale parallel convolutions and/or dilation to capture peak spacing without pooling.

For hyperspectral images, the machine learning models 330 can be configured to implement 2-dimensional (2D) and 1D spectral separability (e.g., through a 2-dimensionl+1-dimensional encoder), using 2D convolution to identify a spatial feature(s) (e.g., a spatial texture feature) and 1D convolution along the spectral axis to identify a chemistry feature (e.g., indicating a chemical element, a chemical compound, and/or the like). In at least some instances, depth wise-separable spectral convolutions can reduce parameter count and/or overfitting.

In some implementations, the machine learning models 330 can facilitate use of physically constrained input representations (e.g., as opposed to raw intensities that violate physical invariances). For example, the machine learning models 330 can be configured to perform log-intensity encoding (e.g., shot-noise stabilization), determine ratio features between known biochemical peaks, and/or treat first/second spectral derivatives as channels. In some implementations, the machine learning models 330 can receive multi-channel input (e.g., where channel 1 is associated with intensity, channel 2 is associated with a first derivative, and channel 3 is associated with second derivative). Physically constrained input representations can force/promote the machine learning models 330 to attend to peak shape rather than absolute scale, improving prediction performed.

In some implementations, the machine learning models 330 can implement spectral attention mechanisms to leverage chemical information that is localized in wavelength bands. More specifically, the machine learning models 330 can determine attention weights over bands and then perform weighted spectral aggregation using the attention weights. In some implementations, the machine learning models 330 can perform group-wise attention over known regions (e.g., fingerprint region) and/or regularize attention for smoothness along a given wavelength(s).

In some implementations, the machine learning models 330 can include physics-informed layers to recognize that received spectra data obeys physical constraints. For example, the machine learning models 330 can enforce (1) non-negativity enforced via ReLU and/or softplus activation functions, (2) peak-shape constraints via convolutional filters initialized to Gaussian/Lorentzian forms, and/or (3) energy conservation constraints across bands. In some implementations, the machine learning models 330 can implement learnable but constrained filters initialized to known Raman peak profiles.

In some implementations, the machine learning models 330 can be configured to implement cross-modal attention, where modalities provide complementary evidence for other modalities to refine identified features represented by either modality (e.g., to produce refined features). For example, the machine learning models 330 can use (1) Raman features to attend to hyperspectral regions and (2) hyperspectral features to attend to Raman peaks. In some implementations, this cross-modal attention can be chemistry guided (e.g., by restricting attention to plausible band correspondences). In some implementations, the machine learning models 330 can perform the cross-modal attention such that attention smoothness (e.g., via kernel smoothing) is narrower (e.g., by a predefined factor) for a Raman modality (e.g., represented by Raman spectrum data) and broader (e.g., by a predefined factor) for a hyperspectral imaging (HSI) modality (e.g., represented by hyperspectral image data).

In some implementations, the machine learning models 330 can be configured to perform shared-latent contrastive learning to overcome, for example, scarcity of labeled multimodal data. For example, the machine learning models 330 can include encoders that are trained such that paired Raman/HIS samples map closely in latent space. The machine learning models 330 can further use contrastive loss (e.g., InfoNCE and/or the like) to perform modality-missing inference and/or to improve generalization.

In some implementations, the machine learning models 330 can use training-time customizations, such as spectral data augmentation, loss function adaptations, and/or self-supervised pretraining. Spectral data augmentation can include, for example, peak shifting, baseline warping, band dropout (e.g., to simulate sensor failure), spectral mixing (e.g., to produce linear combinations), and/or adversarial augmentation (e.g., gradient-based perturbations constrained to spectral smoothness).

In some implementations, the machine learning models 330 can leverage loss function adaptations to account for misclassification having varying degrees of inaccuracy. For example, the machine learning models 330 can be configured to recognize a hierarchy of losses (e.g., species→genus), spectral smoothness regularization, and/or domain-invariance losses (e.g., instrument/day).

In some implementations, to reduce labelling, the machine learning models 330 can use self-supervised pretraining, which can include masked band reconstruction, cross-modal prediction (e.g., between Raman and HIS predictions), and/or spectral ordering prediction.

In some implementations, the machine learning models 330 can apply output-level constraints and/or interpretation through (1) chemistry-aware uncertainty (e.g., using Bayseian neural networks and/or Monte Carlo (MC) dropout to calibrate uncertainty across modalities) and/or (2) interpretability constraints (e.g., by enforcing sparse attention and/or penalizing reliance on non-physics bands).

The output integrator 340 can integrate (e.g., combine, select, and/or etc.) a plurality of outputs from the machine learning models 330 using, for example, voting, weighting, ranking, selection, and/or suppression techniques. Weights associated with the outputs can be dynamically adjusted using performance metrics, confidence scores, confusion matrices, environmental context, and/or adversarial robustness indicators. In some instances, models can be temporarily and/or permanently (e.g., for a given session) excluded based on degradation and/or drift. In some implementations, the output integrator 340 can facilitate voting based on a combination of spectrographics that represent how the machine learning models 330 detect and identify pathogens in real time (or near real time) and in the context of data input, preprocessing, model inference, and/or result integration. Spectrographics can represent, for example, how each spectrum is normalized, how baseline artifacts are suppressed, how per-spectrum predictions are weighted, and/or how final identification is voted and ranked.

With respect to voting, the output integrator 340 can implement replicate-aware voting (e.g., using logit averaging and/or Bayesian updating), where weights can depend on signal-to-noise ratio (SNR), baseline curvature, and/or spike count. In some implementations, the voting can be taxonomy-aware, to suppress implausible species-level predictions early in model execution. Alternatively or in addition, the output integrator 340 can perform SNR-weighted predictions, peak-confidence weighting, modality-confidence weighting (multi-modal), plausibility-constrained ranking, confidence-calibrated ranking, multi-resolution ranking, open-set selection (e.g., based on a reconstruction error threshold), region-of-support selection (e.g., using only reliable spectral regions), progressive evidence accumulation, baseline dominance suppression (e.g., by measuring contribution of low-frequency components and, if dominance exceeds threshold, suppress prediction and request re-acquisition), composite decision pipelines, and/or the like.

In some implementations, the output integrator 340 can align latent spaces of modality-specific encoders (e.g., without sharing encoder weights, to improve data privacy), such as a 1D Raman encoder and a 2D+1D hyperspectral encoder (described herein). In some implementations, the output integrator 340 can include a gating network configured to select and/or weigh modality paths to perform inferencing despite missing modality data and/or degraded sensor conditions. In some implementations, the output integrator 340 can perform confidence-aware routing (e.g., in response to low-SNR Raman data, rely more on HSI data).

In some implementations, the output integrator 340 can perform cross-modal distillation in response to one modality having a higher fidelity. For example, the output integrator 340 can include a high-capacity teacher trained on rich modality data (e.g., HSI data) and distill resulting knowledge to a lighter Raman-only model. As a result, modality-specialized models inherit cross-modal structure.

In some implementations, the output integrator 340 can include a shared encoder associated with hierarchical model heads to account for biological taxonomy hierarchy. For example, the hierarchical model heads can include a genus head and a species head that is conditioned on the genus head. The shared encoder can use a loss function L=Lgenus+αLspecies|genus, such that species classifiers are active only within a predicted genus (e.g., to reduce processor usage, bandwidth usage, and/or memory usage).

In some implementations, the output integrator 340 can implement class-conditioned feature modulation to account for different pathogens expressing different biochemical markers. More specifically, the output integrator 340 can perform feature-wise linear modulation (FILM) based on pathogen class and/or superclass. As a result, the same encoder can adapt internally to multiple pathogen chemistries.

In some implementations, the output integrator 340 can implement one-vs-many anomaly heads to facilitate open-set recognition. For example, confirmed/identified pathogens can be processed using a discriminative classifier (e.g., a more efficient model), and unconfirmed/unidentified pathogens can be processed using a density and/or reconstruction error model (e.g., a less efficient model, which can be used as-needed to conserve compute resource usage).

In some implementations, the output integrator 340 can include domain-specific adapters (e.g., one adapter per region and/or environment, placed after encoders) to account for geography changes that manifest as strain genetics, growth media, and/or environmental background spectra. The domain-specific adapters can reduce full model retraining, conserving compute resource usage.

In some implementations, the output integrator 340 can perform domain-adversarial training to prevent overfitting to region. More specifically, the output integrator 340 can include a geography classifier with gradient reversal, where an encoder learns geography-invariant features. In some implementations, early (e.g., upstream) layers of the encoder are geography-aware, and late (e.g., downstream) layers are geography-invariant.

In some implementations, the output integrator 340 can track geographically aware priors based on some pathogens being region-specific. A prior can include, for example, a Bayesian prior on class probabilities conditioned on region, maintaining discrimination while incorporating epidemiology. In some implementations, the output integrator 340 can include condition-aware normalization layers (e.g., for conditions such as instrument, illumination, sample prep, SNR, and/or etc.) to account for different laser powers, different detectors, and/or etc.

In some implementations, the output integrator 340 can implement a mixture-of-experts by condition (e.g., a first expert for high SNR, a second expert for low SNR, a third expert for wet samples, and a gated output).

In some implementations, the self-calibration heads can account for conditions that drift over time. More specifically, an auxiliary head can predict SNR, baseline curvature, and/or illumination scale, and these predications can feed back into preprocessing parameter selection and/or adaptive routing.

The output integrator 340 can produce a result (e.g., a weighted combination of outputs from the machine learning models 330 and/or an output from a machine learning model(s) selected from the machine learning models 330), which can be stored at the analysis database 350. A user interface (e.g., that is functionally and/or structurally similar to the user interface 122 of FIG. 1) can retrieve the result from the analysis database 350 for use in, for example, clinical diagnostics, public health monitoring, pathogen detection (e.g., in plants/crops, water samples, soil samples, air samples, and/or etc.), and/or the like.

The trainer 360 can be configured to facilitate at least one of training, continual learning, and/or federated learning. For the example, the trainer 360 can be configured to aggregate training datasets from multiple instruments, locations, and/or entities (e.g., organizations). The trainer 360 can train models using centralized learning, continual learning (e.g., to identify new pathogens, media/substrates, instrument firmware, seasonal background shifts, and/or etc.), online learning, and/or federated learning, and the trainer 360 can further facilitate sharing of model updates without transferring raw data, which can improve data privacy and/or security. In some implementations, the trainer 360 can apply adversarial augmentation and/or confounder simulation to improve robustness, as described further below.

Continual learning can include, for example, replay-based continual learning (e.g., using stored cluster centroids rather than raw spectra, conserving memory resources), regularization-based methods (e.g., elastic weight consolidation (EWC), synaptic intelligence (SI), etc.), and/or architecture expansion (e.g., by preserving chemical feature extractors and/or adding lightweight pathogen- or geography-specific heads).

Online learning can include updating the model incrementally (e.g., spectrum-by spectrum and/or batch-by-batch) during operation. More specifically, the trainer 360 can use online calibration heads to keep classifiers fixed and update normalization parameters, illumination correction, and/or baseline alignment, adapting to drift without changing chemistry. In some implementations, the trainer 360 can implement confidence-gate updates to prevent learning from poor and/or corrupted spectra.

In some implementations, the trainer 360 can employ federated learning to, for example, keep sensitive data (e.g., patient spectra data) on-site while a global pathogen encoder learns federatively. Local voting can further incorporate hospital-specific priors to improve performance.

Adversarial augmentation in the context of training machine-learning models on spectral data can include deliberately adding worst-case, but physically plausible perturbations to spectra during training to make the machine learning models 330 more robust. For example, instead of training only on clean or randomly augmented spectra, the trainer 360 can generate small perturbations that are designed to maximally confuse the machine learning models 330 (e.g., using gradient-based methods like a fast gradient signed method (FGSM) and/or projected gradient descent (PGD)). These perturbations simulate challenging conditions such as sensor noise, calibration drift, atmospheric effects, and/or subtle spectral mixing that could cause misclassification.

The trainer 360 can therefore force the machine learning models 330 to learn stable, physically meaningful spectral features rather than weak correlations, improving robustness to real-world variability, domain shift, and adversarial and/or out-of-distribution input, which can be useful at least in hyperspectral and/or remote sensing applications.

Illustrating the trainer 360 in the context of E. coli vs S. aureus classification, the trainer 360 can facilitate model training to identify bacteria (e.g., E. coli vs. S. aureus) from Raman spectra. An original sample can include a Raman spectrum x (intensity vs. wavenumber) with label y=E. coli. An adversarial augmentation can be found by computing a small perturbation that maximally increases classification loss:

$${x}_{a d v} = x + ϵ \cdot sign ( {∇}_{x} ℒ ( f ( x ) , y ) )$$

The perturbation can be constrained to be physically plausible, by having, for example, very small peak intensity changes (simulating laser power fluctuations), slight baseline distortions (simulating fluorescence variation), and/or minor peak broadening or shifts (simulating instrument drift). The trainer 360 can train the machine learning models 330 on both the original and adversarial perturbed spectra, such that the machine learning models 330 learn to rely on robust biochemical Raman signatures (e.g., nucleic acid and protein peaks) rather than unreliable peak heights, improving identification accuracy across instruments, days, and/or sample conditions.

In some implementations, in use during a training phase, the trainer 360 can receive as input data collected via the data acquirer 310 and using multiple spectroscopes. The received data can be labeled (e.g., prior to being received by the trainer 360) based on, for example, bacterial type and/or concentration. In some implementations, the trainer 360 can augment the received data using, for example, statistical variations of the spectral response on a per wavelength basis. In some implementations, in addition to permitting the machine learning models 330 to select features of the received data based on the respective learning processes of the machine learning models 330, the trainer 360 can also instruct the machine learning models 330 to consider predetermined features (e.g., determined based on prior experimentation and/or observation) that are indicators/are sufficiently statistically significant for use in classifying the received data. As used herein, a feature can include and/or be associated with, for example, a peak, peak shift, peak-to-peak ratio, peak-to-trough ratio, first derivative, and/or second derivative, of a spectrum distribution.

The trainer 360 can further validate the machine learning models 330 using, for example, k-fold validation and/or random data splits to define, from the received data, training data and validation data. Alternatively or in addition, the trainer 360 can split the received data based on metadata that represents, for example, geographic location of a sample, a collection device from a plurality of collection devices, and/or the like. In some implementations, the trainer 360 can be configured to validate the machine learning models 330 using blind studies, performing independent verification of sample types using detection techniques different from those performed by the machine learning models 330, such as polymerase chain reaction (PCR) and/or the like. To assess model performance during training and/or validation and perform retraining and/or hyperparameter finetuning as a result, the trainer 360 can be configured to evaluate, for the machine learning models 330, negative predictive values (NPV), positive predictive values (PPV), receiver operating characteristic (ROC) curves, F1 scores, and/or etc.

In use during an inferencing phase, the spectroscopy analysis components 300 can acquire and process data in real time or near real time. In some implementations, at least some of the spectroscopy analysis components 300 can be distributed across devices, edge nodes, and/or cloud servers. In some implementations, the spectroscopy analysis components 300 can facilitate parallel processing, sub 1-second laser integration time (compared to 75-90 second integration time for some known systems), use of both darkfield and brightfield optics to gather data, and/or processing of multimodal data, as described herein.

To further illustrate the spectroscopy analysis components 300 in use, an example application of the spectroscopy analysis components 300 includes analysis of Raman spectroscopy data using a hybrid edge-cloud ensemble of machine learning models. In this example, a Raman spectrometer (e.g., a handheld Raman spectrometer) can acquire spectrum data from a spectrometer(s) via the data acquirer 310. The spectrum data can represent a measurement of, for example, a liquid sample including multiple bacterial pathogens. Initial preprocessing (via the preprocessor 320) and inference using a lightweight neural network (included in the machine learning models 330) can occur on the Raman spectrometer. The Raman spectrometer can transmit the spectra and intermediate embeddings produced by the lightweight neural network to a remote compute device (e.g., a cloud platform), where, for example, gradient-boosted trees and a deep convolutional neural network (CNN) (each included in the machine learning models 330) can perform additional inference. The output integrator 340 (e.g., executed at the remote compute device) can then dynamically weight outputs from the gradient-boosted trees and the deep CNN based on, for example, recent validation performance to identify and quantify, for example, E. coli and/or Salmonella.

As yet another illustration of the spectroscopy analysis components 300 in use, an additional example application of the spectroscopy analysis components 300 includes analysis of hyperspectral images using federated learning. In this example, hyperspectral images of crop leaves can be acquired via the data acquirer 310 across geographically distributed farms. Local random forest and Siamese neural network models can perform inference on-site (e.g., as to the spectroscopy device that implements the data acquirer 310). Updates to the local random forest and Siamese neural network models can be periodically aggregated via the trainer 360 using federated learning to improve detection of fungal and bacterial pathogens while preserving data locality. The output integrator 340 can produce spatially resolved detections by, for example, dynamically selecting the most confident model output (from remaining model outputs) per image region.

The data acquirer 310 can be configured to acquire raw spectra data generated by the Raman spectrometer. This raw spectra data can include, for example, fluorescence backgrounds, shot noise, and/or baseline drift. The preprocessor 320 can therefore apply to the raw spectra data at least one of a baseline correction (e.g., using polynomial and/or rolling-circle methods via the baseline corrector 323, to result in corrected spectrum data), noise smoothing (e.g., Savitzky-Golay filtering via the denoiser 324, to result in denoised spectral data), and/or normalization (e.g., vector and/or total-area normalization via the normalizer 322, to result in normalized spectrum data), to ensure that downstream analyses focus on relevant biochemical peaks rather than instrumentation artifacts.

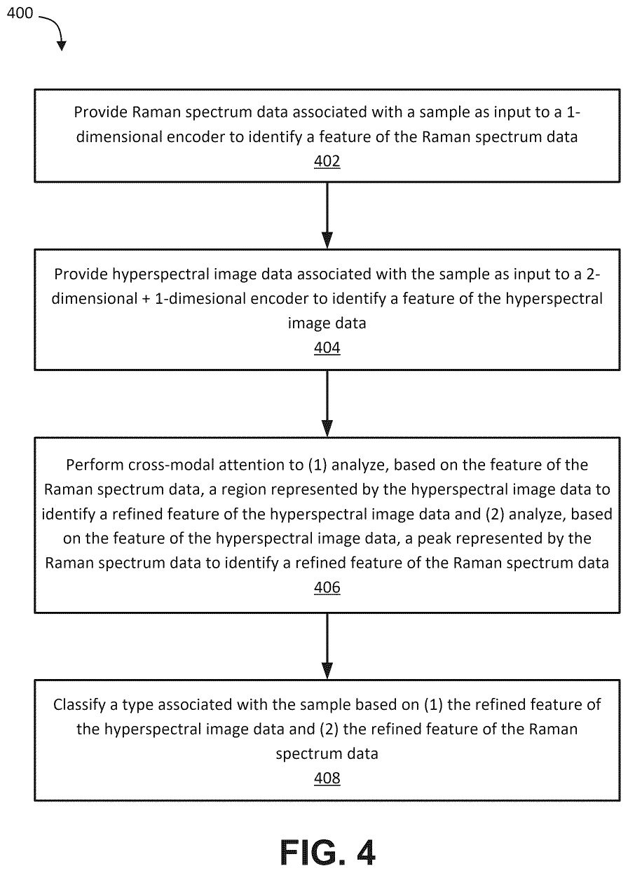

FIG. 4 shows a flow diagram illustrating a method 400 for classifying a type associated with a sample based on refined features of Raman spectrum data and hyperspectral image data, according to an embodiment. In some instances, the method 400 can be implemented by a spectroscopy analysis system (e.g., the spectroscopy analysis system 100 of FIG. 1). Portions of the method 400 can be implemented using a processor (e.g., the processor 220 of FIG. 2) of any suitable compute device (e.g., the compute device 201 of FIG. 2 and/or the compute devices 110 and/or 120 and/or the server 130 of FIG. 1).

The method 400 at 402 includes providing Raman spectrum data associated with a sample as input to a 1-dimensional encoder to identify a feature of the Raman spectrum data. At 404, the method 400 includes providing hyperspectral image data associated with the sample as input to a 2-dimensional+1-dimesional encoder to identify a feature of the hyperspectral image data. Cross-modal attention is performed at 406 to (1) analyze, based on the feature of the Raman spectrum data, a region represented by the hyperspectral image data to identify a refined feature of the hyperspectral image data and (2) analyze, based on the feature of the hyperspectral image data, a peak represented by the Raman spectrum data to identify a refined feature of the Raman spectrum data. A type associated with the sample is classified at 408 based on (1) the refined feature of the hyperspectral image data and (2) the refined feature of the Raman spectrum data.

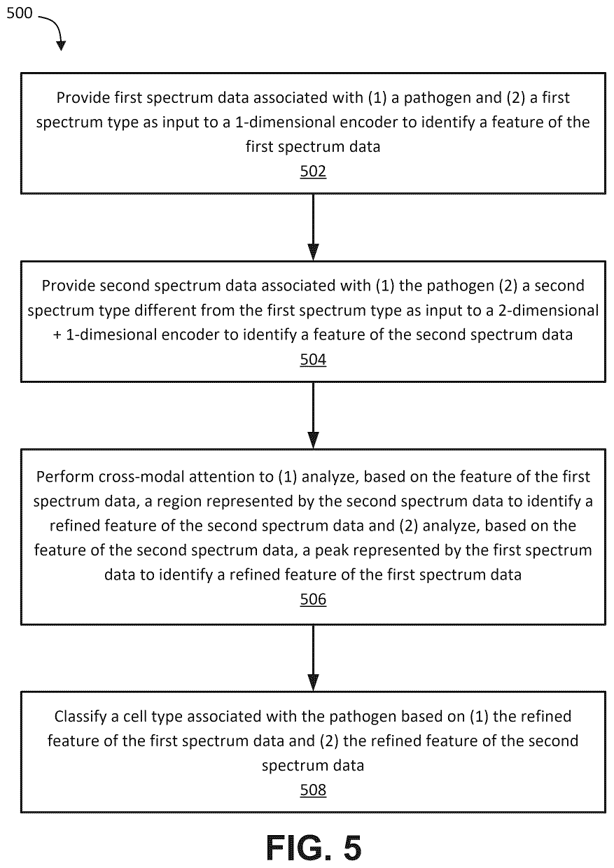

FIG. 5 shows a flow diagram illustrating a method 500 for classifying a cell type associated with a pathogen based on refined features of first and second spectrum data, according to an embodiment. In some instances, the method 500 can be implemented by a spectroscopy analysis system (e.g., the spectroscopy analysis system 100 of FIG. 1). Portions of the method 500 can be implemented using a processor (e.g., the processor 220 of FIG. 2) of any suitable compute device (e.g., the compute device 201 of FIG. 2 and/or the compute devices 110 and/or 120 and/or the server 130 of FIG. 1).

The method 500 at 502 includes providing, via a processor, first spectrum data associated with (1) a pathogen and (2) a first spectrum type as input to a 1-dimensional encoder to identify a feature of the first spectrum data. At 504, the method 500 includes providing, via the processor, second spectrum data associated with (1) the pathogen (2) a second spectrum type different from the first spectrum type as input to a 2-dimensional+1-dimesional encoder to identify a feature of the second spectrum data. Cross-modal attention is performed at 506, via the processor, to (1) analyze, based on the feature of the first spectrum data, a region represented by the second spectrum data to identify a refined feature of the second spectrum data and (2) analyze, based on the feature of the second spectrum data, a peak represented by the first spectrum data to identify a refined feature of the first spectrum data. The method 500 at 508 includes classifying, via the processor, a cell type associated with the pathogen based on (1) the refined feature of the first spectrum data and (2) the refined feature of the second spectrum data.

In some embodiments, a method for detecting and identifying multiple pathogens in the presence of confounding factors includes acquiring sensor data from one or more spectroscopic or imaging modalities and adaptively preprocessing the sensor data. The method further includes applying a plurality of heterogeneous machine learning models to the preprocessed data and dynamically integrating, selecting, weighting, ranking, and/or suppressing outputs of the plurality of machine learning models to generate pathogen detection results.

In some implementations, the preprocessing is dynamically controlled based on inferred noise characteristics or metadata. In some implementations, at least two machine learning models are of different architectural classes. In some implementations, integration weights are adjusted based on historical or real-time performance metrics. In some implementations, the method further includes estimating pathogen concentration, abundance, or confidence. In some implementations, inference is distributed across edge devices and cloud servers. In some implementations, the method further includes continual and/or online learning. In some implementations, models are trained using federated learning without sharing raw sensor data. In some implementations, the method further includes adversarial training and/or confounder simulation.

In some instances, the method can be performed by a system for detecting and identifying multiple pathogens. The system can include one or more sensors, one or more processors, and a non-transitory computer-readable medium storing instructions that cause the processors to perform the method. In some implementations, the processors can dynamically disable and/or suppress individual machine learning models. In some implementations, the system can operate in real time or near real time. In some implementations, the sensor(s) can include a Raman, hyperspectral, fluorescence, and/or FTIR sensor(s).

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages and development tools. Additional examples of computer code include, but are not limited to, control signals, encrypted code, and compressed code.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

The acts performed as part of a disclosed method(s) can be ordered in any suitable way. Accordingly, embodiments can be constructed in which processes or steps are executed in an order different than illustrated, which can include performing some steps or processes simultaneously, even though shown as sequential acts in illustrative embodiments. Put differently, it is to be understood that such features can not necessarily be limited to a particular order of execution, but rather, any number of threads, processes, services, servers, and/or the like that can execute serially, asynchronously, concurrently, in parallel, simultaneously, synchronously, and/or the like in a manner consistent with the disclosure. As such, some of these features can be mutually contradictory, in that they cannot be simultaneously present in a single embodiment. Similarly, some features are applicable to one aspect of the innovations, and inapplicable to others.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Some embodiments described herein relate to a computer storage product with a non-transitory computer-readable medium (also can be referred to as a non-transitory processor-readable medium and/or a machine-readable medium) having instructions or computer code thereon for performing various computer-implemented operations. The computer-readable medium (or processor-readable medium, machine-readable medium, etc.) is non-transitory in the sense that it does not include transitory propagating signals per se (e.g., a propagating electromagnetic wave carrying information on a transmission medium such as space or a cable). The media and computer code (also can be referred to as code) can be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules can include, for example, a processor, a field programmable gate array (FPGA), and/or an application specific integrated circuit (ASIC). Software modules (executed on hardware) can include instructions stored in a memory that is operably coupled to a processor and can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

What is claimed is:

1. A non-transitory, processor-readable medium storing instructions that, when executed by a processor, cause the processor to:
provide Raman spectrum data associated with a sample as input to a 1-dimensional encoder to identify a feature of the Raman spectrum data;
provide hyperspectral image data associated with the sample as input to a 2-dimensional+1-dimesional encoder to identify a feature of the hyperspectral image data;
perform cross-modal attention to (1) analyze, based on the feature of the Raman spectrum data, a region represented by the hyperspectral image data to identify a refined feature of the hyperspectral image data and (2) analyze, based on the feature of the hyperspectral image data, a peak represented by the Raman spectrum data to identify a refined feature of the Raman spectrum data; and
classify a type associated with the sample based on (1) the refined feature of the hyperspectral image data and (2) the refined feature of the Raman spectrum data.

2. The non-transitory, processor-readable medium of claim 1, wherein:
the 1-dimensional encoder includes a convolutional neural network (CNN) configured to perform a plurality of 1-dimensional convolutions using a plurality of kernels, each kernel from the plurality of kernels associated with (1) a different number of wavelength bands and (2) a different feature from a plurality of features that includes the feature of the Raman spectrum data.

3. The non-transitory, processor-readable medium of claim 1, wherein:
the feature of the hyperspectral image data includes a spatial feature and a chemistry feature; and
the 2-dimensional+1-dimesional encoder includes a convolutional neural network (CNN) configured to perform (1) a 2-dimensional convolution on a plurality of pixels represented by the hyperspectral image data to identify the spatial feature and (2) a 1-dimensional convolution on along a spectral axis represented by the hyperspectral image data to identify the chemistry feature.

4. The non-transitory, processor-readable medium of claim 1, wherein:
the feature of the Raman spectrum data includes a single peak feature and a multi-peak feature; and
the 1-dimensional encoder includes a convolutional neural network (CNN) configured to perform (1) a first 1-dimensional convolution using a first kernel associated with a first number of wavelength bands to identify the single peak feature and (2) a second 1-dimensional convolution using a second kernel (a) different from the first kernel and (b) associated with a second number of wavelength bands that is greater than the first number of wavelength bands, to identify the multi-peak feature.

5. The non-transitory, processor-readable medium of claim 1, wherein the instructions to cause the processor to perform the cross-modal attention include instructions to cause the processor to:
perform the cross-modal attention based on (1) a first attention smoothness associated with the hyperspectral image data and (2) a second attention smoothness associated with the Raman spectrum data and narrower than the first attention smoothness.

6. The non-transitory, processor-readable medium of claim 1, wherein at least one of the 1-dimensional encoder or the 2-dimensional+1-dimesional encoder is trained to ignore impulsive noise in at least one of the Raman spectrum data or the hyperspectral image data, using training data having synthetically injected spike data.

7. The non-transitory, processor-readable medium of claim 1, wherein:
the sample includes a pathogen; and
the type includes a cell type associated with the pathogen.

8. The non-transitory, processor-readable medium of claim 1, wherein:
the refined feature of the Raman spectrum data (1) represents at least one of a peak, a peak shift, a peak-to-peak ratio, a peak-to-trough ratio, a first derivative, or a second derivative, of the Raman spectrum data and (2) is discriminative of the type associated with the sample.

9. The non-transitory, processor-readable medium of claim 1, further storing instructions to cause the processor to:
receive raw spectrum data;
perform peak-anchored alignment of the raw spectrum data by identifying an invariant anchor point based on a non-absorbing window represented by the raw spectrum data;
estimate a baseline offset based on the invariant anchor point; and
apply a baseline correction to the raw spectrum data based on the baseline offset to produce at least one of the Raman spectrum data or the hyperspectral image data.

10. The non-transitory, processor-readable medium of claim 1, further storing instructions to cause the processor to:
receive raw spectrum data;
detect a baseline drift in the raw spectrum data based on at least one of a curved baseline, a sloped baseline, a peak asymmetry, or an elevated background, represented by the raw spectrum data;
in response to detecting the baseline drift, define an increased asymmetric least squares (ALS) smoothing parameter value; and
apply ALS smoothing to the raw spectrum data based on the increased ALS smoothing parameter value, to produce at least one of the Raman spectrum data or the hyperspectral image data.

11. The non-transitory, processor-readable medium of claim 1, further storing instructions to cause the processor to:
receive raw spectrum data;
mean-center the raw spectrum data to produce mean-center data;
generate covariance matrix data based on the mean-center data;
perform eigen-decomposition on the covariance matrix data to identify at least one eigenvector; and
reduce a dimensionality of the raw spectrum data based on the at least one eigenvector, to produce at least one of the Raman spectrum data or the hyperspectral image data.

12. A method, comprising:
providing, via a processor, first spectrum data associated with (1) a pathogen and (2) a first spectrum type as input to a 1-dimensional encoder to identify a feature of the first spectrum data;
providing, via the processor, second spectrum data associated with (1) the pathogen (2) a second spectrum type different from the first spectrum type as input to a 2-dimensional+1-dimesional encoder to identify a feature of the second spectrum data;
performing, via the processor, cross-modal attention to (1) analyze, based on the feature of the first spectrum data, a region represented by the second spectrum data to identify a refined feature of the second spectrum data and (2) analyze, based on the feature of the second spectrum data, a peak represented by the first spectrum data to identify a refined feature of the first spectrum data; and
classifying, via the processor, a cell type associated with the pathogen based on (1) the refined feature of the first spectrum data and (2) the refined feature of the second spectrum data.

13. The method of claim 12, wherein:
the 1-dimensional encoder includes a convolutional neural network (CNN) configured to perform a plurality of 1-dimensional convolutions using a plurality of kernels, each kernel from the plurality of kernels associated with (1) a different number of wavelength bands and (2) a different feature from a plurality of features that includes at least one of the feature of the first spectrum data or the feature of the second spectrum data.

14. The method of claim 12, wherein:
the second spectrum data includes hyperspectral image data;
the feature of the hyperspectral image data includes a spatial feature and a chemistry feature; and
the 2-dimensional+1-dimesional encoder includes a convolutional neural network (CNN) configured to perform (1) a 2-dimensional convolution on a plurality of pixels represented by the hyperspectral image data to identify the spatial feature and (2) a 1-dimensional convolution on along a spectral axis represented by the hyperspectral image data to identify the chemistry feature.

15. The method of claim 12, wherein:
at least one of the feature of the first spectrum data or the feature of the second spectrum data includes a single peak feature and a multi-peak feature; and
the 1-dimensional encoder includes a convolutional neural network (CNN) configured to perform (1) a first 1-dimensional convolution using a first kernel associated with a first number of wavelength bands to identify the single peak feature and (2) a second 1-dimensional convolution using a second kernel (a) different from the first kernel and (b) associated with a second number of wavelength bands that is greater than the first number of wavelength bands, to identify the multi-peak feature.

16. The method of claim 12, wherein at least one of the 1-dimensional encoder or the 2-dimensional+1-dimesional encoder is trained to ignore impulsive noise in at least one of the first spectrum data or the second spectrum data, using training data having synthetically injected spike data.

17. The method of claim 12, wherein:
at least one of the refined feature of the first spectrum data or the refined feature of the second spectrum data represents at least one of a peak, a peak shift, a peak-to-peak ratio, a peak-to-trough ratio, a first derivative, or a second derivative, that is discriminative of the cell type associated with the pathogen.

18. The method of claim 12, further comprising:
receiving, via the processor, raw spectrum data;
performing, via the processor, peak-anchored alignment of the raw spectrum data by identifying an invariant anchor point based on a non-absorbing window represented by the raw spectrum data;
estimating, via the processor, a baseline offset based on the invariant anchor point; and
applying, via the processor, a baseline correction to the raw spectrum data based on the baseline offset to produce at least one of the first spectrum data or the second spectrum data.

19. The method of claim 12, further comprising:
receiving, via the processor, raw spectrum data;
detecting, via the processor, a baseline drift in the raw spectrum data based on at least one of a curved baseline, a sloped baseline, a peak asymmetry, or an elevated background, represented by the raw spectrum data;
in response to detecting the baseline drift, increasing, via the processor, an asymmetric least squares (ALS) smoothing parameter value to produce an increased ALS smoothing parameter value; and
applying, via the processor, ALS smoothing to the raw spectrum data based on the increased ALS smoothing parameter value, to produce at least one of the first spectrum data or the second spectrum data.

20. The method of claim 12, further comprising:
receiving, via the processor, raw spectrum data;
mean-centering, via the processor, the raw spectrum data to produce mean-center data;
generating, via the processor, covariance matrix data based on the mean-center data;
performing, via the processor, eigen-decomposition on the covariance matrix data to identify at least one eigenvector; and
reducing, via the processor, a dimensionality of the raw spectrum data based on the at least one eigenvector, to produce at least one of the first spectrum data or the second spectrum data.
